(19) United States
(12) Patent Application Publication
(10) Pub. No.: US 2022/0125747 A1
(43) Pub. Date: Apr. 28, 2022

(54) APPLICATION OF ACTIVE INGREDIENT WGX50 IN ZANTHOXYLUM BUNGEANUM EXTRACT

(71) Applicants: Shanghai Jiao Tong University, Shanghai (CN); Yantai Zhiyi Medicine Technology Co., Ltd, Yantai City (CN); Nantong Haizhi Pharmaceutical Technology Co., Ltd, Nantong City (CN)

(72) Inventors: Dongqing Wei, Shanghai (CN); Heng Wang, Shanghai (CN)

(21) Appl. No.: 17/570,584

(22) Filed: Jan. 7, 2022

Related U.S. Application Data
(63) Continuation of application No. PCT/CN2021/100141, filed on Jun. 15, 2021.

(30) Foreign Application Priority Data
Jun. 17, 2020 (CN) 202010551170.4

(51) Int. Cl. A61K 31/165; A61K 36/75

(52) U.S. Cl. CPC A61K 31/165; A61K 36/75

(57) ABSTRACT

An application method of the WGX50 of active ingredient in Zanthoxylum bungeanum extract is provided, after testing the cytotoxicity of keratinocytes and melanocytes with different concentrations of WGX50, conducting a moisturizing gene test based on keratinocytes, a whitening gene test based on melanocytes and an anti-inflammatory test based on UVB stimulation of keratinocytes. First discovers that WGX50 has the efficacys of moisturizing, whitening and anti-inflammation, and the discovery of the new efficacies of WGX50 provides a potential scheme for preparing multifunctional cosmetics, opens up new ideas for preparing cosmetics, and contributes to the realization of higher commercial value.

### TECHNICAL FIELD

The invention relates to the field of biotechnology, and more particularly to an application method of WGX50 of active ingredient in Zanthoxylum bungeanum extract.

### BACKGROUND

Zanthoxylum bungeanum is rich in chemical components, mainly including alkaloid, amide, lignin, coumarin, flavonoid, volatile oil and fatty acid, etc., and Zanthoxylum bungeanum has some efficacy, such as easing pain, anaesthesia, insect killing, anticancer and antioxidation. At the same time, Zanthoxylum bungeanum, as a common medicinal and edible traditional Chinese medicine, is often used as an aromatic condiment in daily life. These properties make Zanthoxylum bungeanum an ideal source for seeking plant extracts or effective active ingredients.

The discovered WGX50 ((E)-N[2-(3,4-dimethoxyphenyl) ethyl]-3-phenylacrylamide) of active ingredient in Zanthoxylum bungeanum extract has the specific structure as follows:

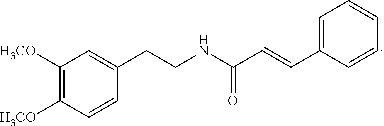 [SMILES: COc1ccc(CCNC(=O)/C=C/c2ccccc2)cc1OC]

Chinese patent application No. CN201210034380.1 (corresponding to Chinese patent publication No. CN102579272A), entitled “Application of GX50 in Cosmetic Composition and Cosmetic Composition Containing GX50” mentions that GX50, as the main active ingredient in the preparation of cosmetic compositions, has the functions of promoting collagen synthesis, restoring skin elasticity and inhibiting the formation of wrinkles. These functions of GX50 in preparing cosmetic compositions in the form of cosmetic additives indicate that WGX50 of active ingredient in Zanthoxylum bungeanum extract, has excellent performance in anti-aging.

The problem to be solved by the invention is whether WGX50 of active ingredient in Zanthoxylum bungeanum extract has other undisclosed new applications, so as to be used in cosmetics preparation, developing new ideas for cosmetics preparation and to realize higher commercial value.

### SUMMARY

In order to solve the existing technical problem, the objective of the invention is to provide a new application of WGX50 of active ingredient in the Zanthoxylum bungeanum extract.

In order to achieve the above objective, the invention adopts the following technical schemes:

a new application method of WGX50 of active ingredient in Zanthoxylum bungeanum extract.

The inventors conduct the following experiments:

1. Moisturizing gene test based on keratinocytes. First, the cytotoxicity of keratinocytes is tested, and then the experimental design is carried out, and a blank control group and a sample group are set up. The detection indexes are AQP3 (aquaporin), CD44 (cell-surface glycoproteins), Filaggrin (protein), claudin-1 (tight junction protein) and HAS2 (hyaluronic acid synthetase). RNA extraction, reverse transcription and fluorescence quantitative PCR are carried out, and the results are calculated by 2^{-ΔΔCT} method. The results show that compared with the blank control group, WGX50 may significantly promote the expression of AQP3 gene in keratinocytes.

2. Whitening gene test based on melanocytes. First, the cytotoxicity of melanocytes is tested, and then the experimental design is carried out, and a blank control group and a sample group are set up. The detection indexes are MC1R (melanocortin receptor), MITF (melanogenesis transcription factor), TYR (melanin synthesis and transport related gene) and TYP-1 (tyrosinase related protein). RNA extraction, reverse transcription and fluorescence quantitative PCR are carried out, and the results are calculated by 2^{-ΔΔCT} method. The results show that compared with the blank control group, WGX 50 may significantly inhibit the expression of MC1R, MITF, TYR and TYP-1 genes.

3. Anti-inflammatory test based on UVB (ultraviolet radiation b) stimulation of keratinocytes. The blank control (oil-soluble), solvent control (oil-soluble), positive control and sample groups are grouped in the experiment. The detection indexes are TNFα (a pro-inflammatory factors, the NF-κB signaling pathway mediates and initiates the transcription of TNFα (abbreviation for tumor necrosis factor alpha), and after TNFα is synthesized, it binds to its receptor and in turn activates the NF-κB signaling pathway) and IL1-α (a pro-inflammatory factor being non-hydrophobic polypeptide, which is secreted out of cells after cell membrane damage, and then the pro-inflammatory factor binds to its corresponding receptor IL1R and activates NF-κB to induce inflammatory cascade reaction), the detection and analysis are performed according to the operating manual of the TNFα and IL1-α kits.

The results of TNFα detection show that compared with the solvent control group, the secretion of WGX50 TNFα decreases significantly.

The results of IL-1α (abbreviation for interleukin-1α) detection show that compared with the solvent control group, the secretion of WGX50 IL-1α decreases significantly.

The above experiments show that the new applications of WGX50 of active ingredient in Zanthoxylum bungeanum extract, have the functions of moisturizing, whitening and anti-inflammation.

Furthermore, WGX50 may significantly promote the expression of AQP3 gene in keratinocytes, and WGX50 has the efficacy of moisturizing.

Furthermore, WGX 50 may significantly inhibit the expression of MC1R, MITF, TYR and TYP-1 genes, and WGX50 has the efficacy of whitening.

Furthermore, the secretion of WGX50 TNFα decreases significantly, and the secretion of WGX50 IL-1α decreases significantly. WGX50 has the efficacy of anti-inflammation.

The beneficial effects of the invention are as follows: the invention firstly discovered the new functions of moisturizing, whitening and anti-inflammation of the WGX50 of active ingredient in Zanthoxylum bungeanum extract besides anti-aging. The discovery of the new functions of WGX50 provides a potential scheme for the preparation of multifunctional cosmetics, helps develop new ideas for preparing cosmetics, and contributes to the realization of higher commercial value.

### BRIEF DESCRIPTION OF THE FIGURES

FIG. 1 is a diagram showing the cell viability change trend of keratinocytes of the invention.

FIG. 2 is a diagram showing the cell morphology of the keratinocytes of the invention.

FIG. 3 is a diagram showing the cell viability change trend of the melanocytes of the invention.

FIG. 4 is a diagram showing the change trend of moisturizing genes in keratinocytes of the invention.

FIG. 5 is a diagram showing the melanocyte whitening gene change trend of the invention.

FIG. 6 is a diagram showing the TNFα content change trend of the invention.

FIG. 7 is a diagram showing the IL-1α content change trend of the invention.

### DESCRIPTION OF THE INVENTION

The invention will be described in detail with reference to the drawings and specific embodiments below.

The cells used in this test are keratinocytes and melanocytes, and all cells are produced by Guangdong Biocell Biotechnology Co., Ltd, in China. WGX50 powder is prepared according to the method disclosed in the Chinese patent application No. 201110439656.X (corresponding to Chinese patent publication No. CN103130674A) entitled “Chemical Synthesis Method of Potential Medicine gx50, gx51, gx52, and gx180 for the preventing and treating the Alzheimer's disease”.

The reagents used are PBS (Boster), MTT(Sigma), DMSO(Sigma), DMEM (Guangdong Biocell), NBS (Sijiqing), Medium-254(Gibco), HMGS(Gibco), KC2500 (Guangdong Biocell), Dexamethasone (Sigma), RNAiso Plus (TaKaRa), reverse transcription kit (TaKaRa), fluorescent dye (TaKaRa), TNFα kit (Abcam) and IL1-α kit (Abcam).

The main equipment used includes CO₂ incubator (Thermo), clean bench (Airtech), inverted microscope (Olympus), microoscillator (Kylin-Bell), enzyme-labeled instrument (BioTek) and flow cytometer (Beckman).

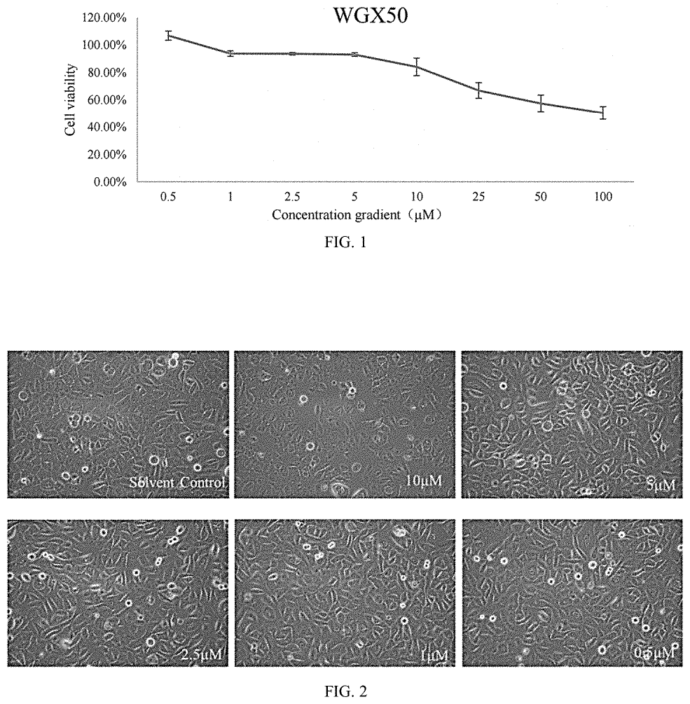

Embodiment 1, cytotoxicity test based on keratinocytes. Set up 8 concentration gradients, and set up 3 repeating holes under each concentration. At the same time, solvent control hole, zero adjustment hole (KC2500) and positive control hole (PC, 4% Dimethyl sulfoxide, DMSO) are set up in the experiment. After incubation for 24 hours, the cell viability is detected by MTT method, and the maximum safe concentration of cell administration is screened. The MTT test results are shown in Table 1, in which the GraphPad Prism Program software is used for mapping, and T-test statistical analysis is used among all groups. P-value<0.05 indicates significant difference, and P-value<0.01 indicates extremely significant difference. The change trend of cell viability is shown in FIG. 1. According to the results of MTT assay, the concentration near the inflection point of cell vitality is selected, and after incubation and culture for 24 hours, the cell morphology is observed under the inverted microscope and photographed (20×), and the morphological detection results are shown in FIG. 2.

TABLE 1
Toxicity test results of keratinocytes

| | | Concentration gradient (μM) | | | | | | | | | |
|---|---|---|---|---|---|---|---|---|---|---|---|
| | | \textcircled{1} | \textcircled{2} | \textcircled{3} | \textcircled{4} | \textcircled{5} | \textcircled{6} | \textcircled{7} | \textcircled{8} | PC | Control |
| | | 0.5 | 1 | 2.5 | 5 | 10 | 25 | 50 | 100 | 4% DMSO | / |
| Cell | Mean | 106.81% | 93.72% | 93.60% | 93.01% | 84.00% | 66.77% | 57.35% | 50.36% | 30.27% | 100% |
| vitality | SD | 3.29% | 1.96% | 0.89% | 1.29% | 6.36% | 5.71% | 6.02% | 4.62% | 2.09% | 1.88% |
| | P-value | 0.036 | 0.016 | 0.006 | 0.006 | 0.014 | 0.001 | 0.000 | 0.000 | 0.000 | / |

According to MTT and morphological results, WGX50 shows no cytotoxicity to keratinocytes in the concentration range of 5 μM.

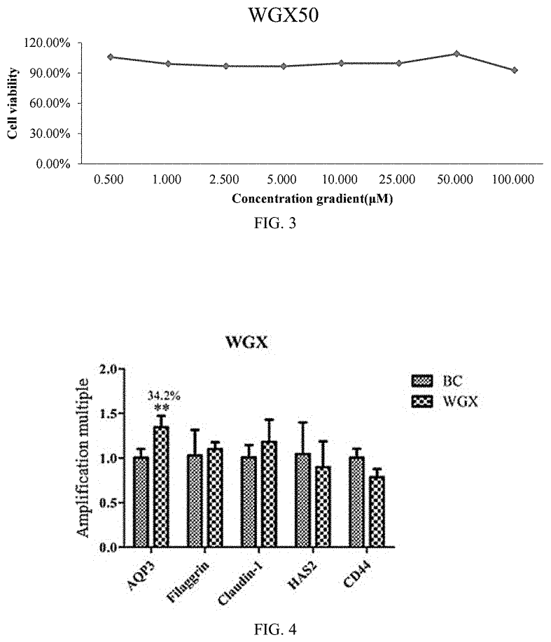

Embodiment 2, cytotoxicity test based on melanocytes. Set up 8 concentration gradients, and set up 3 repeating holes under each concentration. At the same time, solvent control well, Medium-254 well and positive control well (PC, 8% DMSO) are set up in the experiment. After incubation for 24 hours, the cell viability is detected by MTT method, and the maximum safe concentration of cell administration is screened. The results of MTT assay are shown in Table 2, and the change trend of cell viability is shown in FIG. 3.

TABLE 2
Toxicity test results of melanocytes

| | | Concentration gradient (μM) | | | | | | | | | |
|---|---|---|---|---|---|---|---|---|---|---|---|
| | | \textcircled{1} | \textcircled{2} | \textcircled{3} | \textcircled{4} | \textcircled{5} | \textcircled{6} | \textcircled{7} | \textcircled{8} | PC | Control |
| | | 0.5 | 1 | 2.5 | 5 | 10 | 25 | 50 | 100 | 8% DMSO | / |
| Cell | Mean | 105.78% | 99.02% | 96.68% | 95.56% | 99.51% | 99.51% | 108.86% | 96.62% | 24.23% | 100.00% |
| vitality | SD | 3.33% | 1.07% | 4.62% | 2.62% | 4.82% | 3.79% | 7.78% | 3.15% | 6.00% | 5.68% |
| | P-value | 0.196 | 0.783 | 0.476 | 0.394 | 0.914 | 0.907 | 0.186 | 0.121 | 0.000 | / |

According to the results of MTT, WGX50 shows no cytotoxicity to melanocytes in the concentration range of 100 μM.

Embodiment 3, moisturizing gene test based on keratinocytes. The experimental design of moisturizing gene test based on keratinocytes is shown in Table 3.

TABLE 3
Experimental design of moisturizing gene test

| | Sample | | | | | |
|---|---|---|---|---|---|---|
| | information | | Detection Systems | | | |
| Experi- | | Sample | | | | Incu- |
| ment | Sample | concen- | Detection | Detection | Detection | bation |
| grouping | name | tration | Indicator | model | method | time |
| Blank | BC | / | AQP3, | Keratino- | qRT-PCR | 24 h |
| control | | | CD44, | cytes | | |
| group | | | Filaggrin, | | | |
| Sample | WGX50 | 5 μM | claudin-1, | | | |
| group | | | HAS2 | | | |

AQP3 aquaporin in the detection index, located on the cell membrane, has the function of transporting water and plays an important role in the process of skin moisturizing.

Gene detection process: after incubation for 24 hours, the cells are sampled, and RNA extraction, reverse transcription and fluorescence quantitative PCR detection are carried out according to the kit instruction, and the results are calculated by 2^{-ΔΔΔCT} method. When statistical analysis is carried out by T-Test method, significance is represented by *, P-value<0.05 is represented by *, and P-value<0.01 is represented by * *.

The detection results are shown in Table 4, and the trend of gene change is shown in FIG. 4.

TABLE 4
Test results of moisturizing gene

| | BC (Blank | | | | | | Increased |
|---|---|---|---|---|---|---|---|
| | control group) | | | WGX50 | | | ratio |
| Sample | | | P- | | | P- | (compared |
| name | Mean | SD | value | Mean | SD | value | to BC) |
| AQP3 | 1.004 | 0.099 | / | 1.345 | 0.127 | 0.005** | 34.20% |
| Filaggrin | 1.029 | 0.287 | / | 1.101 | 0.076 | 0.646 | 7.20% |
| Claudin-1 | 1.007 | 0.139 | / | 1.182 | 0.249 | 0.267 | 17.50% |
| HAS2 | 1.046 | 0.354 | / | 0.898 | 0.289 | 0.543 | −14.70% |
| CD44 | 1.004 | 0.101 | / | 0.785 | 0.092 | 0.101 | −21.90% |

Compared with the blank control group, WGX50 significantly promotes the expression of AQP3 gene in keratinocytes (P-value<0.01).

Embodiment 4, whitening gene test based on melanocytes. The experimental design of whitening gene test based on melanocytes is shown in Table 5.

TABLE 5
Experimental design of whitening gene test

| | Sample | | | | | |
|---|---|---|---|---|---|---|
| | information | | Detection Systems | | | |
| Experi- | | Sample | | | | Incu- |
| ment | Sample | concen- | Detection | Detection | Detection | bation |
| grouping | name | tration | Indicator | model | method | time |
| Blank | BC | / | MC1R, | Melano- | qRT-PCR | 24 h |
| control | | | MITF, | cytes | | |
| group | | | TYR, | | | |
| Sample | WGX50 | 100 μM | TRP-1 | | | |
| group | | | | | | |

Gene detection process: after 24 hours of incubation, the cells are sampled, and RNA extraction, reverse transcription and fluorescence quantitative PCR detection are carried out according to the kit instructions, and the results are calculated by 2^{-ΔΔΔCT} method. When statistical analysis is carried out by T-Test method, significance is represented by *, P-value<0.05 is represented by *, and P-value<0.01 is represented by * *.

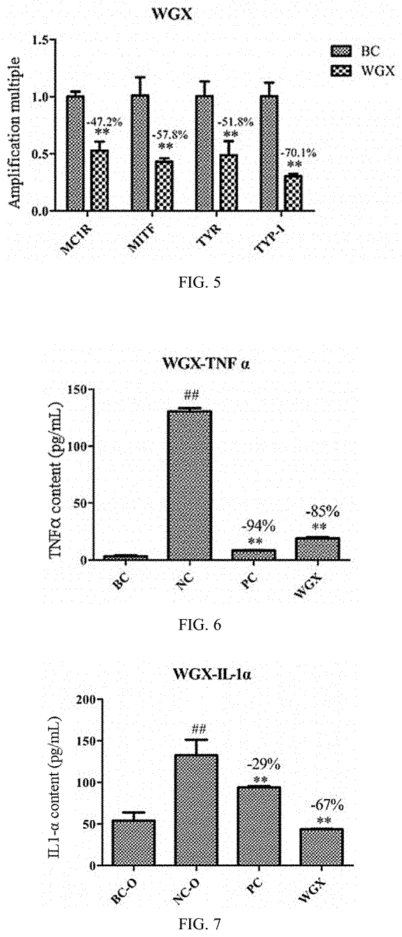

The detection results are shown in Table 6, and the trend of gene change is shown in FIG. 5.

TABLE 6
Results of whitening gene detection

| | BC (Blank | | | | | | Increased |
|---|---|---|---|---|---|---|---|
| | control group) | | | WGX50 | | | ratio |
| Sample | | | P- | | | P- | (compared |
| name | Mean | SD | value | Mean | SD | value | to BC) |
| MC1R | 1.001 | 0.043 | / | 0.528 | 0.079 | 0.000** | 47.2% |
| MITF | 1.01 | 0.16 | / | 0.432 | 0.029 | 0.000** | 57.8% |
| TYR | 1.006 | 0.127 | / | 0.488 | 0.122 | 0.000** | 51.8% |
| TRP-1 | 1.005 | 0.118 | / | 0.304 | 0.022 | 0.000** | 70.10% |

Compared with the blank control group, WGX50 significantly inhibits the expression of MC1R, MITF, TYR and TYP-1 genes in melanocytes (P-value<0.01).

Embodiment 5, anti-inflammatory test of keratinocytes stimulated by UVB. The experimental design of anti-inflammatory test of keratinocytes stimulated by UVB is shown in Table 7.

TABLE 7
Experimental design of anti-inflammatory test

| | Sample information | | | Detection Systems | | | | |
|---|---|---|---|---|---|---|---|---|
| Experiment | Sample | Sample | | | Detection | Detection | Detection | Incubation |
| grouping | name | concentration | | Stimulus | Indicator | model | method | time |
| Blank | BC-O | DMSO (0.1%) | | / | TNFα, | Keratinocytes | ELISA | 24 h |
| control | | | | | IL1-α | | | |
| (oil soluble) | | | | | | | | |
| Solvent | NC-O | | | UVB | | | | |
| control | | | | (300 ml/cm²) | | | | |
| (oil soluble) | | | | | | | | |
| Positive | PC | 100 | μg/mL | | | | | |
| control | (Dexamethasone) | | | | | | | |
| Sample | WGX50 | 5 | μM | | | | | |
| group | | | | | | | | |

TNFα and IL1-α expression detection process: after 24 hours of incubation, the cell culture supernatant is collected, and the detection and analysis are performed according to the operating instructions of the TNFα and IL1-α kit. When using the T-Test method for statistical analysis, the NC group is compared with the BC group, and the significance is indicated by #, P-value<0.05 is indicated as #, P-value<0.01 is indicated as ##; sample group and positive control group are compared with NC group, and significance is represented by *, P-value<0.05 is represented by *, P-value<0.01 is represented by * *.

The results of TNFα measurement are shown in Table 8, and the change trend is shown in FIG. 6.

TABLE 8
Detection results of TNFα

| | Average | | | Decreased ratio |
|---|---|---|---|---|
| | concentration | | | (compared |
| Sample name | (pg/mL) | SD | P-value | to NC) |
| BC | 3.13 | 0.95 | / | / |
| (Blank control group) | | | | |
| NC | 130.46 | 2.85 | 0.00 | / |
| (Solvent control group) | | | | |
| PC | 8.34 | 0.41 | 0.00** | 94% |
| (Positive control group) | | | | |
| WGX50 | 18.97 | 1.04 | 0.00** | 85% |

Compared with the blank control group, the TNFα secretion in the positive control group is significantly increased (P-value<0.01), indicating that the UVB stimulation conditions in this experiment are effective. Compared with the solvent control group, the secretion of TNFα decreases significantly (P-value<0.01) at the concentration of 100 μg/mL dexamethasone, which shows that this experiment is effective.

Compared with the positive control group, the secretion of WGX50 TNFα decreases significantly (P-value<0.01).

The detection results of IL-1α a are shown in Table 9 and the change trend is shown in FIG. 7.

TABLE 9
Detection results of IL-1α

| | Average | | | Decreased ratio |
|---|---|---|---|---|
| | concentration | | | (compared |
| Sample name | (pg/mL) | SD | P-value | to NC) |
| BC | 53.86 | 10.05 | / | / |
| (Blank control group) | | | | |
| NC | 132.52 | 18.75 | 0.00## | / |
| (Solvent control group) | | | | |
| PC | 93.97 | 1.62 | 0.02* | 29% |
| (Positive control group) | | | | |
| WGX50 | 43.65 | 0.59 | 0.00** | 67% |

Compared with the blank control group, the IL-1α secretion in the positive control group significantly increases (P-value<0.05), indicating that the UVB stimulation conditions in this experiment are effective. Compared with the solvent control group, the secretion of IL-1α decreases significantly (P-Value<0.05) at the concentration of dexamethasone of 100 μg/mL, which indicates that this experiment is effective.

Compared with the positive control group, the secretion of WGX50 IL-1α decreases significantly (P-Value<0.01).

What is claimed is:

1. An application method of (E)-N-[2-(3,4-dimethoxyphenyl) ethyl]-3-phenylacrylamide (WGX50) of active ingredient in Zanthoxylum bungeanum extract, wherein the WGX50 of active ingredient in Zanthoxylum bungeanum extract is applied for one or more selected from the group consisting of moisturizing, whitening and anti-inflammation.

2. The application method of WGX50 of active ingredient in Zanthoxylum bungeanum extract according to claim 1, wherein the WGX50 of active ingredient is applied in promoting expression of AQP3 gene in keratinocytes, and the WGX50 of active ingredient has an efficacy of the moisturizing.

3. The application method of WGX50 of active ingredient in Zanthoxylum bungeanum extract according to claim 1, wherein the WGX 50 of active ingredient is applied in inhibiting expressions of MC1R, MITF, TYR and TYP-1 genes, and the WGX50 of active ingredient has an efficacy of the whitening.

4. The application method of WGX50 of active ingredient in Zanthoxylum bungeanum extract according to claim 1, wherein the WGX50 of active ingredient is applied in decreasing secretions of TNFα and IL-1α, and the WGX50 of active ingredient has an efficacy of the anti-inflammation.

5. The application method of WGX50 of active ingredient in Zanthoxylum bungeanum extract according to claim 1, wherein the WGX50 of active ingredient has the following structure: 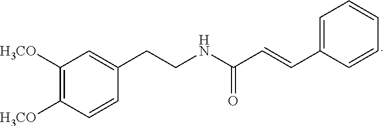 [SMILES: COc1ccc(CCNC(=O)/C=C/c2ccccc2)cc1OC]
